(19) United States
(12) Patent Application Publication
(10) Pub. No.: US 2020/0075128 A1
(43) Pub. Date: Mar. 5, 2020

(54) Analyzing High Dimensional Data Based on Hypothesis Testing for Assessing the Similarity between Complex Organic Molecules Using Mass Spectrometry

(71) Applicant: ScinoPharm Taiwan, Ltd., Shan-Hua (TW)

(72) Inventors: Lung-Cheng Lin, Tainan (TW); Pao-Chi Liao, Tainan (TW)

(73) Assignee: ScinoPharm Taiwan, Ltd.

(21) Appl. No.: 16/530,544

(22) Filed: Aug. 2, 2019

Related U.S. Application Data
(60) Provisional application No. 62/726,342, filed on Sep. 3, 2018.

(51) Int. Cl. G16B 40/10; G01N 33/68; G16C 60/00; G16C 20/70

(52) U.S. Cl. CPC G16B 40/10; G16C 20/70; G16C 60/00; G01N 33/6848

(57) ABSTRACT

The present invention developed a hypothesis testing approach to analyze the high-dimensional LC-MS data to assess the extent of similarity between a reference drug and generics.

### CROSS-REFERENCES TO RELATED APPLICATIONS

This application claims priority from U.S. Provisional Patent Application Ser. No. 62/726,342, which was filed on Sep. 3, 2018. The entire content of this provisional application is incorporated herein by reference.

### BACKGROUND OF THE INVENTION

Glatiramer acetate (GA), a complex heterogeneous mixture of synthetic polypeptides, has been approved as an immunomodulatory drug by the US Food and Drug Administration (FDA) for the treatment of relapsing-remitting multiple sclerosis, the most common disabling neurological disorder of young adults.

Glatiramer acetate (GA) is the active ingredient of COPAXONE® (Teva Pharmaceutical Industries Ltd., Israel), comprises the acetate salts of a synthetic polypeptide mixture containing four naturally occurring amino acids: L-glutamic acid, L-alanine, L-tyrosine, and L-lysine, with a reported average molar fraction of 0.141, 0.427, 0.095, and 0.338, respectively. The average molecular weight of COPAXONE® is between 4,700 and 11,000 daltons. In controlled clinical trials, Copaxone has been demonstrated to have a 75% reduction in relapse rate over 2 years and significantly reduce progression of disability in multiple sclerosis with long-term efficacy, safety, and tolerability. The extensive use and relatively high cost of Copaxone leads to an evolving need for development of other generic versions of GA to increase affordability and access to this medication.

GA is one kind of non-biological complex drugs (NBCDs). Over the years, a robust regulatory system for development of generic versions of small molecule medicines, which can be fully identified and characterized, has been well-established using the concept of pharmaceutical equivalence and bioequivalence. However, the regulatory policies and analytical approaches for biologicals and NBCDs remain under development. Since NBCDs are usually synthesized complex macromolecules/mixtures that cannot be fully characterized, they are suggested to be evaluated based on the “similarity” with the reference listed drug, like “biosimilar approaches” for biologicals.

More than 10³⁶ possible theoretical sequences exist in GA, which makes its components neither fully identifiable nor quantifiable even by the up-to-date analytical techniques. Therefore, no two GA can ever be proved “identical”. Various chemical analyses, including molecular mass distribution profiling by gel permeation chromatography, peptide mapping by capillary electrophoresis, relative amino acid levels at the N-termini by Edman degradation, secondary structure characterization by circular dichroism, and proteolytic digests profiling by reverse-phase high-performance liquid chromatography (RP-HPLC), have been conducted to compare GA drugs.

### SUMMARY OF THE INVENTION

The present invention developed a hypothesis testing approach to analyze the high-dimensional LC-MS data to assess the extent of similarity between a reference drug and generics. One characteristic of our proposed hypothesis testing approach is to consider the differences in all data points between two sample groups. Besides, additional resampling technique can introduce robust inference procedures, even for a small number of samples. These characteristics lead to the robust results obtained from this approach.

### BRIEF DESCRIPTION OF THE DRAWINGS

FIG. 1 (a) illustrates base peak chromatograms of 7 replicate samples of one batch of Copolymer-1 sample.

FIG. 1 (b) illustrates base peak chromatograms of 7 replicate samples of one lot of negative control.

FIG. 1 (c) illustrates base peak chromatograms of 10 lots of Copaxone and one batch of Copolymer-1 sample.

FIG. 1 (d) illustrates base peak chromatograms of 10 lots of Copaxone and one lot of negative control. The chromatograms show several distinct peaks between the Copaxone and negative control in the first 7 min.

FIG. 2 (a) illustrates a distribution of 10,000 bootstrap estimates derived from the sum of squared deviations test procedure for comparisons between Copaxone and Copaxone.

FIG. 2 (b) illustrates a distribution of 10,000 bootstrap estimates derived from the sum of squared deviations test procedure for comparisons between Copaxone and Copolymer-1 sample.

FIG. 2 (c) illustrates a distribution of 10,000 bootstrap estimates derived from the sum of squared deviations test procedure for comparisons between Copaxone and negative control.

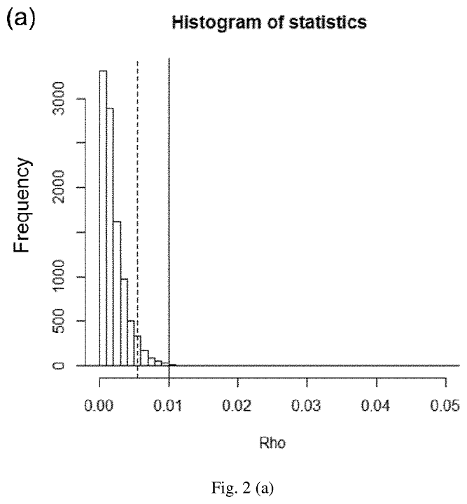

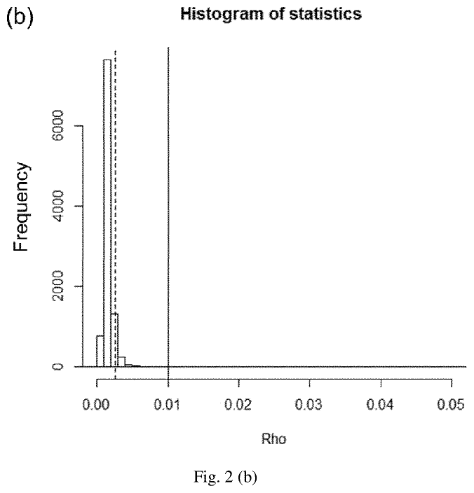

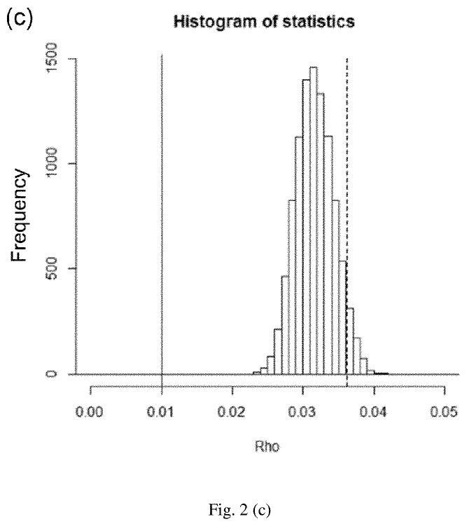

The dash lines on FIGS. 2 (a)-FIG. 2(c) indicate the 95th percentile estimates, and the solid lines indicate the critical values.

### DETAILED DESCRIPTION OF PREFERRED EMBODIMENTS OF THE INVENTION

The phrase “hypothesis testing” as used herein refers to a statistical test used to determine whether the hypothesis assumed for the sample of data stands true for the entire population or not.

The expression “non-biological complex drugs (NBCDs)” as used herein refers to a type of drug with following properties: a) encompassing a complex multitude of closely related structure; b) the properties cannot be fully revealed by physicochemical analysis; c) the entire multitude is the active pharmaceutical ingredient, and d) the consistent, rigorously controlled manufacturing process is essential to reproduce the product.

“Random copolymer drugs” as used herein refers to a drug that is generated from coplymerization process based on the reaction kinetics of chemicals or monomers.

“Polypeptide mixture” as used herein refers to a mixture contains various polypeptides.

“Copolymer mixture” as used herein refers to a mixture containing coplymer.

“Polypeptides” as used herein refers to peptides with short chains of amino acid monomers linked by peptide (amide) bonds.

“Compelex organic molecule” as used herein refers to a polymer-like molecule. Listed reference drug or generic version is digested by Lys-C and followed by UPLC/HILIC-MS analysis. Features in the LC-MS data, identified by the software, such as Progenesis QI for Proteomics software, that can be matched to values in the in-house database were considered to be potential active ingredient of drugs and were further submitted to one developed hypothesis testing approach, sum of squared deviations test, which can process these high-dimensional LC-MS data and evaluate the similarity/difference between sample groups.

The present invention has developed a hypothesis testing approach to assess the similarity between samples. Before performing hypothesis testing on the data points, data points are resampled by resampling technique such as bootstrapping, to regenerate the data points based on the assumption that a statistic can best be assessed by referencing the data it is derived from and is typically used to assess the stability of a statistic or estimate. After obtaining the resampled dataset, we developed a statistical hypothesis testing to compare two datasets. The null hypothesis (H₀) is assumed to be that there are differences between two data sets. The alternative hypothesis (Hₐ) is assumed to be that there is no difference between two data sets, which we conclude when H₀ is rejected. This strategy is to perform hypothesis testing on LC-MS data to determine the similarity/difference of potential active ingredients between two random copolymer drugs, such as peptide drugs. It can also be used to quickly check the lot-to-lot variation in the production process. In principle, this approach also can be applied to non-biological complex drugs (NBCDs) sharing the same characteristics that consist of a multitude of closely related structures, and their properties cannot be fully characterized by physicochemical analysis.

Random copolymer drugs are classified as one kind of non-biological complex drugs (NBCDs) defined as: a) encompassing a complex multitude of closely related structure; b) the properties cannot be fully revealed by physicochemical analysis; c) the entire multitude is the active pharmaceutical ingredient and d) the consistent, rigorously controlled manufacturing process is essential to reproduce the product. Over the years, a robust regulatory system for development of generic versions of small molecule medicines, which can be fully identified and characterized, has been well-established using the concept of pharmaceutical equivalence and bioequivalence. However, the regulatory policies and analytical approaches for biologics and NBCDs remain under development. Since NBCDs are mostly synthesized complex macromolecules/mixtures whose total chemical structure cannot be fully characterized, they are suggested to be evaluated based on the “similarity” with the reference-listed drug, such as “biosimilar approaches” for biologics. No two copolymer drugs can ever be proved “identical”. Various chemical analyses, including molecular mass distribution profiling by gel permeation chromatography, peptide mapping by capillary electrophoresis, relative amino acid levels at the N-termini by Edman degradation, secondary structure characterization by circular dichroism, and proteolytic digests profiling by reverse-phase high-performance liquid chromatography (RP-HPLC), have been conducted to compare glatiramer acetate (GA) drugs. Recently, FDA agency has proposed a molecular fingerprinting approach, including liquid chromatography coupled with mass spectrometry (LC-MS), nuclear magnetic resonance (NMR), and asymmetric field flow fractionation coupled with multi-angle light scattering (AFFF-MALS), to distinguish analytical differences between complex mixtures of peptide chains from GA and non-GA compounds. The study also evaluated the methods' ability to detect analytical differences in the mixtures by applying the statistical analyses to the MS and AFFF-MALS data. However, in that approach, the number of data points (266) was too low to meet these (>1000) suggested by the FDA.

### Example 1

### Sample Preparation

Copolymer-1 (20 mg, purchased from Sigma-Aldrich (St. Louis, Mo.)) or GA (20 mg, ScinoPharm Taiwan Ltd.) was dissolved in 1 mL mannitol (40 mg/mL) at the same concentration as Copaxone, and 7 replicate samples of Copolymer-1 or GA were prepared from 30 μL of the solution. Ten samples were prepared from 30 μL of each lot of Copaxone. For digestion, 45 μL of distilled deionized water (ddH₂O), 18 μL of ammonium bicarbonate (24 mg/mL, adjusted pH 8.40), and 15 μL of Lys-C (0.2 g/L) were added to each sample. These samples were incubated at 37° C. for 16 hours in a water bath. After incubation, 10 μL trifluoroacetic acid (0.1%, v/v) and 118 μL acetonitrile (100%) were added to stop the reaction. These samples were filtered through a hydrophilic polyvinylidene fluoride membrane filter with pore size 0.22 μm (Millipore, Billerica, Mass.). Before UPLC-MS analysis, the samples were stored at −20° C.

### Example 2

High-Dimensional LC-MS Data Generated from Copolymer-1 Samples

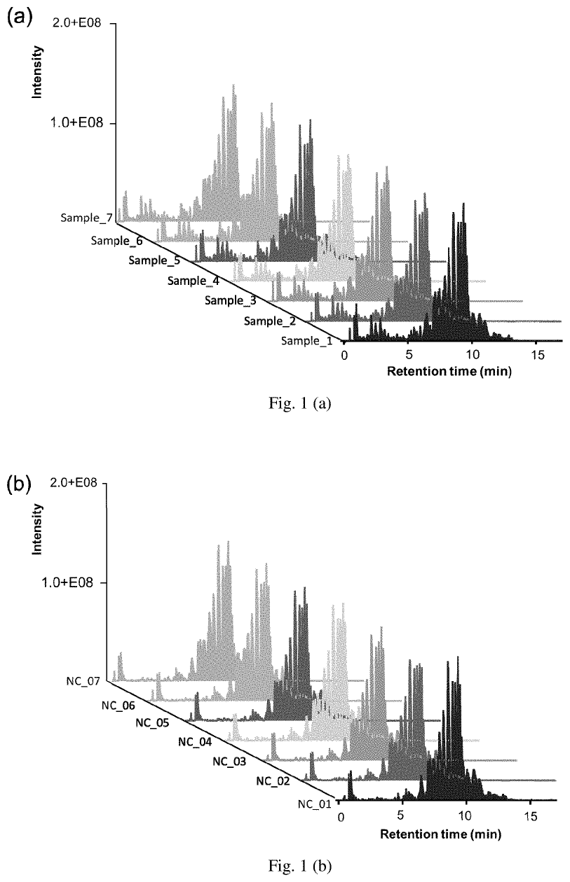

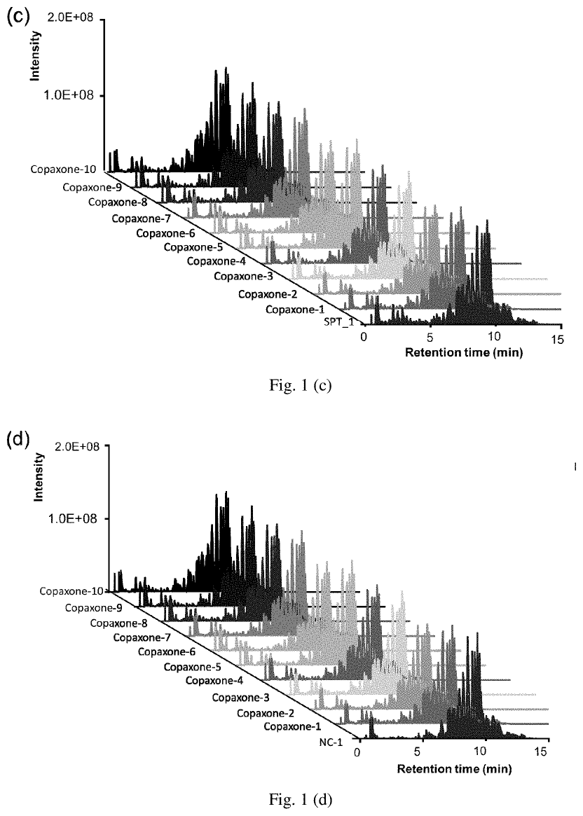

The LC/MS data of the 7 replicate samples from 2 different sources of Copolymer-1 runs, including Copolymer-1 samples and negative control (NC), both look similar (FIGS. 1a Copolymer-1 samples and 1b negative control), indicating the great reproducibility between their individual 7 replicates. Aligning the LC-MS data of Copolymer-1 samples and 10 lots of Copaxone got an average score larger than 95%, implying that the Copolymer-1 samples and Copaxone have similar digested peptide composition. This can also be observed from the LC/MS data among these 11 runs (FIG. 1c). There are several distinct peaks existed in the first 7 min (FIG. 1d) while comparing the 10 lots of Copaxone with one replicate of negative control, where the Copolymer-1 had negligible peaks within this region, suggesting that certain digested peptides were detected only in Copaxone but not in the negative control.

### Example 3

Evaluation of Similarity by Hypothesis Testing

A statistical hypothesis test is a method of statistical inference and commonly applied to comparison of two or more data sets. In the test method, the statistical hypothesis is a testable hypothesis that is based on the basis of observing a process that is modeled via a set of random variables. We developed a hypothesis testing approach to analyze the high-dimensional LC-MS data to assess the extent of similarity between the reference drug and generics. One characteristic of our proposed hypothesis testing approach is to consider the differences in all data points between two sample groups.

To first evaluate the feasibility of this approach, 10 lots of Copaxone were randomly separated into two groups with 5 lots each and their data points were used for the developed sum of squared deviations test. The was \hat{ρ}_{(95%)} (p-value<0.01) showing that H₀ was rejected and different lots of Copaxone were significantly similar (FIG. 2a). We further applied the sum of squared deviations test to Copaxone and Copolymer-1 samples, the estimated \hat{ρ}_{(95%)} was 0.0026 (p-value<0.0001) (FIG. 2b), leading to the rejection of H₀ and suggesting that Copaxone and one batch of Copolymer-1 sample were significantly similar. Comparing Copaxone and the negative control, the estimated \hat{ρ}_{(95%)} was 0.029 (p-value=0.994) (FIG. 2c), which was greater than the critical value, resulting in accepting H₀, and there was evidence to claim that Copaxone and the negative control exhibited differences. These results showed that the developed sum of squared deviations test can be used to assess the similarity between two Copolymer-1 sample groups and was validated by the negative control sample.

A shown in these examples, we developed a hypothesis testing approach on the multivariate (high-dimensional) LC-MS data to assess the extent of similarity between the Copaxone and generics with statistically significance. The statistical significance is used to determine the difference between two groups with probability. In other words, the sameness of profiles between two groups can be determined based on a user setting value.

What is claimed is:

1. A method for characterizing and classifying a sample of a complex organic molecule comprising:
subjecting the sample to mass spectrometry to produce a mass spectrum and analyzing the mass spectrum using a statistic method, wherein the statistic method is hypothesis testing.

2. The method of claim 1 wherein the complex organic molecule is selected from the group consisting of peptides, peptide mixtures, polypeptide mixtures, proteins, protein mixtures, biologics, biosimilars, and combinations thereof.

3. The method of claim 1 wherein the complex organic molecule is a polypeptide mixture.

4. The method according to claim 1, wherein the method comprises:
(a) digesting or decomposing the sample with an appropriate enzyme or chemical to fragments;
(b) analyzing the fragments directly by the mass spectrometry to produce the mass spectrum; and
(c) analyzing the mass spectrum by the hypothesis testing to classify and distinguish different samples.

5. The method according to claim 4, wherein the appropriate enzyme is Lys-C, Trypsin or any other enzymes capable of digesting the sample.

6. The method according to claim 5, wherein the appropriate enzyme is Lys-C.

7. The method according to claim 4, wherein the chemical used to decompose the sample is selected from the group consisting of organic or inorganic acids or bases.

8. The method according to claim 1, wherein the complex organic molecule is a copolymer mixture.

9. The method according to claim 1, wherein the complex organic molecule is glatiramer acetate.

10. The method according to claim 4, wherein the mass spectrometry is LC-MS.

11. A method for analyzing a sample by mass spectrometry comprising:
(a) providing a mixture of polypeptides standard and a mixture of polypeptides sample;
(b) respectively digesting the sample and mixture of polypeptides standard with an appropriate enzyme or chemical;
(c) respectively subjecting the digested mixture of polypeptides sample and mixture of polypeptides standard directly to mass spectrometric analysis to produce two mass spectra; and
(d) comparing and analyzing the two mass spectra by hypothesis testing approach.

12. The method of claim 11 wherein wherein the mixture of polypeptides is glatiramer acetate.

13. The method according to claim 11, wherein the mass spectrometry is LC-MS.

14. A process for preparing a drug product or pharmaceutical composition containing glatiramer acetate, comprising:
(a) polymerizing N-carboxy anhydrides of L-alanine, g-benzyl L-glutamate, trifluoroacetic acid protected L-lysine and L-tyrosine to generate a protected copolymer; reacting protected copolymer with hydrobromic acid to form trifluoroacetyl glatiramer acetate and treating said trifluoroacetyl glatiramer acetate with aqueous piperidine solution to generate a testing sample glatiramer acetate; and purifying the testing sample glatiramer acetate;
(b) analyzing the purified glatiramer acetate test sample and a glatiramer acetate reference standard by using mass spectrometry and hypothesis testing approach.

15. The process according to claim 14, wherein the step of analyzing comprises:
(1) respectively digesting the test sample and reference standard with an appropriate enzyme or chemical;
(2) respectively subjecting the test sample and reference standard directly to mass spectrometry analysis to produce two mass spectra; and
(4) comparing and analyzing the two mass spectra by hypothesis testing approach to determine similarity between the test sample and reference standard sample.

16. The process according to claim 15, wherein the appropriate enzyme is Lys-C, Trypsin or any other enzymes capable of digesting the sample.

17. The method according to claim 15, wherein the appropriate enzyme is Lys-C.

18. The method according to claim 15, wherein the chemical used to decompose the sample is selected from the group consisting of organic or inorganic acids or bases.

19. The method according to claim 15, wherein the mass spectrometry is LC-MS.

20. The method according to claim 15 wherein if the similarity between the test sample and the standard sample is not acceptable, then the method comprises further steps of re-adjusting the conditions of polymerizing, conducting the polymerizing under the re-adjusted conditions, and then conducting the analyzing step again to ensure that the glatiramer acetate is acceptably similar to the reference standard under related requirements.

21. The method of claim 21 wherein the related requirements are made by a government authority or a commercial orgniaization.
